(19) United States
(12) Patent Application Publication
(10) Pub. No.: US 2018/0046769 A1
(43) Pub. Date: Feb. 15, 2018

(54) EMERGENCY RESPONSE KIT

(71) Applicant: DLH, Inc., Long Branch, NJ (US)

(72) Inventor: Edward James O'DONNELL, Morganville, NJ (US)

(21) Appl. No.: 15/677,590

(22) Filed: Aug. 15, 2017

Related U.S. Application Data
(60) Provisional application No. 62/375,403, filed on Aug. 15, 2016.

(51) Int. Cl. G06F 19/00; A61F 17/00

(52) U.S. Cl. CPC G06F 19/34; A61F 17/00; G06F 15/00

(57) ABSTRACT

Emergency response kits are provided herein. In some embodiments, an emergency response kit includes a container having an interior volume; a plurality of equipment kits disposed within the interior volume, wherein each equipment kit contains equipment tailored to a predetermined emergency scenario; and wherein each equipment kit includes an identifying mark that is different from identifying marks of other ones of the plurality of equipment kits; and an instruction device configured to output instructions regarding use of the equipment inside of the plurality of equipment kits.

### CROSS-REFERENCE TO RELATED APPLICATIONS

This application claims the benefit of U.S. Provisional Patent Application No. 62/375,403, filed with the United States Patent Office on Aug. 15, 2016, which is herein incorporated by reference in its entirety.

### FIELD

Embodiments of the present disclosure generally relate to an emergency response kit.

### BACKGROUND

Due the large number of incidents such as accidents, terrorism, violent crimes, and other traumatic incidents around the world, many entities (e.g., government, law enforcement, etc.) rely on the public's assistance in protecting the general public. One specific field in which such entities may rely on public assistance is the medical field since a victim may lose critical time awaiting the arrival of first responders.

Accordingly, the inventor has devised emergency response kit.

### SUMMARY

Embodiments of emergency response kits and methods of use thereof are provided herein. In some embodiments, an emergency response kit includes a container having an interior volume; a plurality of equipment kits disposed within the interior volume, wherein each equipment kit contains equipment tailored to a predetermined emergency scenario; and wherein each equipment kit includes an identifying mark that is different from identifying marks of other ones of the plurality of equipment kits; and an instruction device configured to output instructions regarding use of the equipment inside of the plurality of equipment kits.

In some embodiments, an emergency response kit includes a box having an interior volume; a plurality of containers disposed within the interior volume, wherein each container contains equipment tailored to a predetermined emergency scenario, and wherein each container has a color that is different from colors of other ones of the plurality of containers; and an audio device configured to output audio instructions regarding use of the equipment inside of the plurality of containers, wherein the audio device includes a plurality of pressable buttons, wherein the plurality of pressable buttons each have a color corresponding to a color of one of the plurality of containers, and wherein upon selection of a pressable button, the audio device plays audio instructions regarding use of the equipment inside one of the plurality of containers associated with the pressable button.

In some embodiments, a method of using an emergency response kit includes opening the emergency response kit; selecting one of a plurality of equipment kits disposed within the emergency response kit based on an emergency; pressing a button on an audio device of the emergency response kit corresponding to the selected equipment kit, wherein upon pressing of the button, the audio device plays audio instructions regarding use of equipment inside of the selected equipment kit; and using the equipment to treat a victim of the emergency.

Other and further embodiments of the present disclosure are described below.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts an isometric view of an open emergency response kit in accordance with some embodiments of the present disclosure.

FIG. 2 depicts a schematic side view of an audio device of an emergency response kit in accordance with some embodiments of the present disclosure.

FIG. 3 depicts an isometric view of a closed emergency response kit in accordance with some embodiments of the present disclosure.

FIG. 4 is a flowchart illustrating a method of using an emergency response kit in accordance with some embodiments of the present disclosure.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Emergency response kits are provided herein. The inventive emergency response kit advantageously enables any competent individual to provide aid to a victim of an emergency situation by providing equipment tailored to a specific emergency situation and guidance on how to use the equipment.

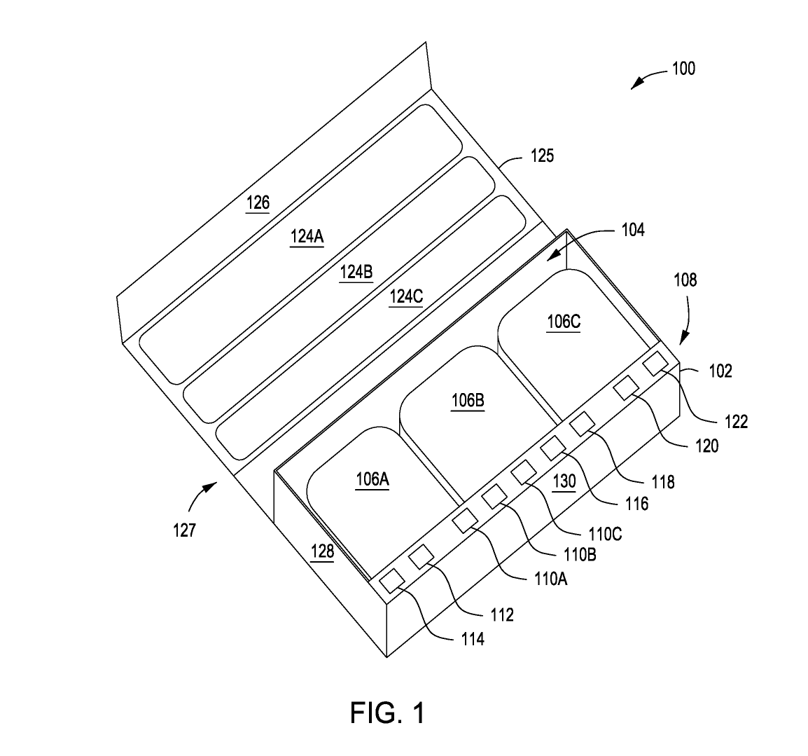

FIG. 1 depicts an isometric view of an emergency response kit 100 in accordance with some embodiments of the present disclosure. In some embodiments, the emergency response kit 100 includes a container 102 having an interior volume 104, a plurality of equipment kits 106A-C disposed within the interior volume 104, and an instruction device 108 coupled to the container 102 and configured to output instructions regarding use of the equipment inside of the plurality of equipment kits 106A-C. FIG. 1 shows the container 102 in an open configuration.

Each of plurality of equipment kits 106A-C contains equipment (not shown) tailored to a predetermined emergency scenario. For example, a first equipment kit 106A may be a compression dressing kit having a compression dressing and, for example, nitrile gloves; a second equipment kit 106B may be a hemostatic dressing kit having a hemostatic dressing, a compression dressing, and nitrile gloves; and the third equipment kit 106C may be a tourniquet kit having a tourniquet and nitrile gloves. However, these are just examples. Each of the plurality of equipment kits 106A-C may contain any equipment suitable for treating a victim of a given emergency. In some embodiments, the plurality of equipment kits 106A-C are bags. In some embodiments, the plurality of equipment kits 106A-C are alternatively boxes. In some embodiments, the emergency response kit 100 may include additional items that may be useful in an emergency situation such as, for example, a lighting device (e.g., a flashlight, a flare, a glow rod, etc) that can be taken out of the container 102, trauma shears, a writing tool (e.g., pen, marker, pencil, etc.), a notepad on which the user can write useful information for first responders, and a biohazard bag in which the user can discard any used equipment.

Each of the plurality of equipment kits 106A-C also includes an identifying mark such as, for example, color. In such an embodiment, each equipment kit has a color that is different from colors of the other equipment kits. By way of example only, the first equipment kit 106A may be green, the second equipment kit 106B may be yellow, and the third equipment kit 106C may be red. To ensure that the instruction device 108 outputs the correct instructions, the instruction device 108 may include a plurality of user-selectable input elements 110A-C corresponding to respective ones of the plurality of equipment kits 106A-C having the same identifying mark as the plurality of equipment kits 106A-C to indicate an association between ones of the plurality of user-selectable input elements 110A-C and corresponding ones of the plurality of equipment kits 106A-C. For example, if the first, second, and third equipment kits 106A-C are green, yellow, and red, respectively, then first, second, and third user selectable input elements 110A-C are also green, yellow, and red, respectively. As such, if a user needs to use the equipment in the second equipment kit 106B, then the user can easily recognize that the second user selectable input element 110B is associated with the second equipment kit 106B. When the user actuates any one of the plurality of user selectable input elements, instructions for how to use the associated equipment kit will be output. Although color has been used as an example, any other symbol, number, letter, etc., or combinations thereof, may be used as an identifying mark on the equipment kits and their corresponding user selectable input elements.

In some embodiments, the instruction device 108 is an audio device configured to play pre-recorded audio instructions regarding use of one of the plurality of equipment kits 106A-C when a corresponding one of the plurality of user-selectable input elements 110A-C is selected. In some embodiments, the plurality of user-selectable input elements 110A-C are pressable buttons. In some embodiments, the instruction device 108 is part of a video display device configured to play pre-recorded audio and video instructions regarding use of one of the plurality of equipment kits 106A-C when a corresponding one of the plurality of user-selectable input elements 110A-C is selected. In some embodiments, the instruction device 108 is a touchscreen and the plurality of user-selectable input elements 110A-C are visual buttons on the touch-screen.

In some embodiments, the instruction device 108 is configured to output instructions in a plurality of user-selectable languages. For example, the instruction device 108 may include one or more user-selectable buttons 112 which a user may select to choose a language in which the instructions are output. In some embodiments, the instruction device 108 may further include other user-selectable buttons to facilitate control of playback of the instructions. For example, the instruction device 108 may include a power toggle button 114, rewind and fast forward buttons 116, 118, volume up and volume down buttons 120, 122, and the like. That is, the instruction device 108 may include all the input commands of a conventional playback device.

In some embodiments, the emergency response kit 100 may include a plurality of written instructions 124A-C also corresponding to ones of the plurality of equipment kits 106A-C. The plurality of written instructions 124A-C may be written on a bottom surface 125 of a top 127 of the container 102. In such an embodiment, each of the plurality of written instructions 124A-C will also include the identifying mark (e.g., color) associating ones of the plurality of written instructions 124A-C with corresponding ones of the plurality of equipment kits 106A-C. The language in which the plurality of written instructions 124A-C are written will depend on which country in which the emergency response kit 100 is installed. In some embodiments, the written instructions 124A-C may include pictures in addition to text to illustrate use of the equipment. In some embodiments, additional instructions or information may be written on surfaces 126, 128, and/or 130 such as, for example, instructions regarding how to operate the instruction device 108. In some embodiments, the bottom surface 125 may be a display screen, which automatically turns on upon opening of the container 102. In such an embodiment, the display screen may display a plethora of information such as, but not limited to, the written instructions 124A-C, pre-recorded videos demonstrating how to use the equipment in each of the plurality of equipment kits 106A-C, and/or a live video feed from a remote facility.

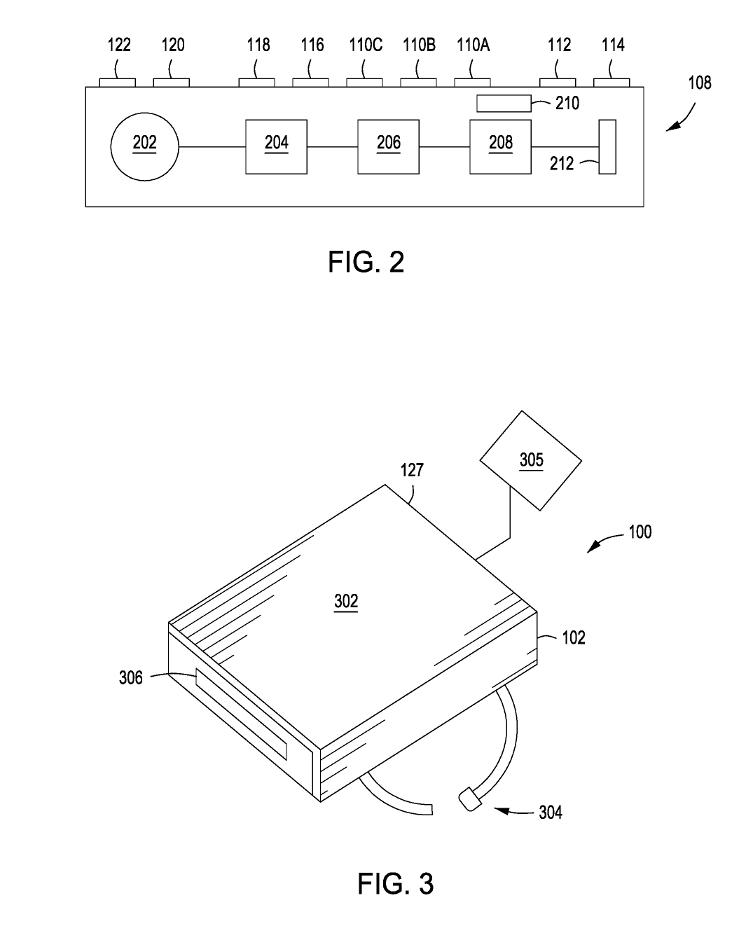

FIG. 2 depicts a schematic side view of the instruction device 108 in accordance with at least some embodiments of the present disclosure. In some embodiments, the instruction device 108 may include a speaker 202, a communication device 204 configured to enable a user to communicate with one or more predetermined parties in real time, a computer-readable medium 206 (e.g., electronic storage device) on which the instructions and contact information for the one or more predetermined parties are stored, a tracking device 208 configured to wirelessly send a location of the emergency response kit 100 to a third party (for example, GPS coordinates, street address, or the like), and a power supply 212 to supply power to elements of the emergency response kit requiring power. The communication device 204 may include, for example, a receiver and transmitter capable of facilitating wireless communication (e.g., via cellular towers) or wired communication. The tracking device 208 may include, for example, a geolocation sensor capable of communicating with a global navigation satellite system (GNSS) to pinpoint the geographical location of the emergency response kit 100 and transmit the location to a predetermined location or facility. In such an embodiment, the contact information may include user-selectable (e.g., buttons, touch-screen options, etc.) to allow the user to choose who to contact. In some embodiments, the power supply 212 may be one or more batteries or a plug that can be coupled to an external power source (e.g., solar panel 305 discussed below) In some embodiments, the instruction device 108 may also include an input/output port 210 to allow the manufacturer to program the instruction device 108 or charge the batteries if the power supply 212 is one or more batteries. In some embodiments, the instruction device 108 may include electrical connections (not shown) coupling the instructions device 108 to a monitor, as discussed above, to power the monitor and display pre-recorded video stored on the computer-readable medium 206.

FIG. 3 depicts an isometric view of the emergency response kit 100 in a closed configuration in accordance with at least some embodiments of the present disclosure. In some embodiments, the emergency response kit may include further information and/or instructions written on an upper surface 302 of the top 127. In some embodiments, the emergency response kit 100 may be coupled to a solar panel 305 to power the emergency response kit 100 and/or to recharge batteries in the instruction device. In some embodiments, the emergency response kit 100 may also include a mounting element 304 configured to allow the emergency response kit 100 to be mounted to a desired structure. For example, the mounting element 304 may be a strap with a buckle to allow the emergency response kit 100 to be mounted to a tree or pole. However, the mounting element 304 may be any suitable apparatus configured to facilitate the mounting of the emergency response kit 100 to any desired structure. In some embodiments, the emergency response kit 100 may further include an identifying element 306 such as for example, a light, which can be used to locate the kit in low light situations (e.g., power outage).

Although the above description has been made with specific examples, the inventive emergency response kit may include additional elements/devices configured to enable a user to administer aid to a victim of an emergency. For example, the emergency response kit 100 may further include attachable indicators (e.g., flags) to allow first responders to prioritize which victims need assistance first. In some embodiments, the emergency response kit 100 may turn on automatically upon opening of the top 127 as opposed to requiring the user to actuate the power toggle button 114. Similarly, in some embodiments, the emergency response kit 100 may include sensors (e.g., light sensors, weight sensors, etc.) to detect when one of the equipment kits 106A-C has been removed from the container 102 and instruct the instruction device 108 to play the instructions corresponding to the equipment kit that has been removed. In such embodiments, the emergency response kit 100 may power down if a predetermined period of inactivity has been detected.

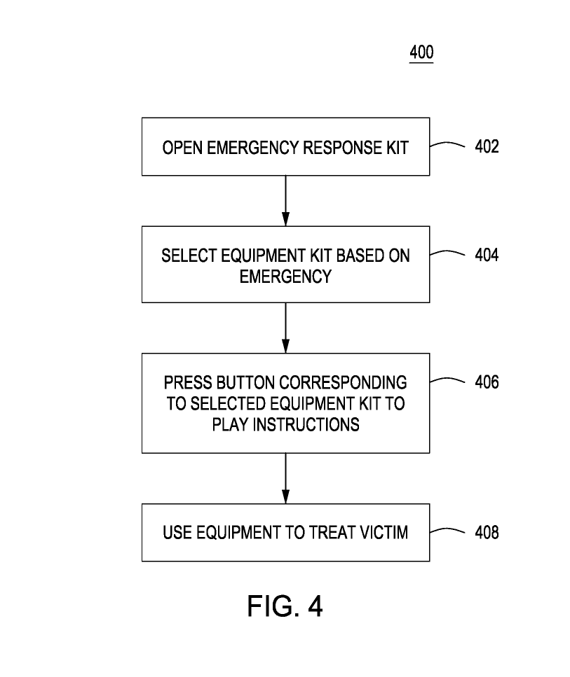

FIG. 4 is a flowchart illustrating a method 400 of using an emergency response kit (e.g., emergency response kit 100) in accordance with some embodiments of the present disclosure. At 402, a user opens the emergency response kit 100. The emergency response kit 100 may be a box or any other similar container. At 404, the user selects one of the plurality of equipment kits 106A-C based on the emergency at hand. At 406, the user presses a button (e.g., 110A-C) on the instruction device 108 (e.g., audio device) corresponding to the selected equipment kit. Upon pressing the button, the instruction device 108 plays pre-recorded audio instructions regarding the use of the equipment inside of the selected equipment kit. At 408, the user uses the equipment per the audio instructions to treat a victim of the emergency.

While the foregoing is directed to embodiments of the present disclosure, other and further embodiments of the disclosure may be devised without departing from the basic scope thereof.

1. An emergency response kit, comprising:
a container having an interior volume;
a plurality of equipment kits disposed within the interior volume, wherein each equipment kit contains equipment tailored to a predetermined emergency scenario, and wherein each equipment kit includes an identifying mark that is different from identifying marks of other ones of the plurality of equipment kits; and
an instruction device configured to output instructions regarding use of the equipment inside of the plurality of equipment kits.

2. The emergency response kit of claim 1, wherein the instruction device includes a plurality of user-selectable input elements corresponding to the plurality of equipment kits, wherein each of the plurality of user-selectable input elements includes the identifying mark to indicate an association between ones of the plurality of user-selectable input elements to corresponding ones of the plurality of equipment kits, and wherein upon selection of one of the plurality of user-selectable input elements, the instruction device outputs instructions regarding use of the equipment inside a corresponding one of the plurality of equipment kits.

3. The emergency response kit of claim 2, wherein the identifying mark is a color, and wherein each user-selectable input element has the same color as a corresponding one of the plurality of equipment kits.

4. The emergency response kit of claim 2, wherein the instruction device is an audio device configured to play audio instructions regarding use of one of the plurality of equipment kits when a corresponding one of the plurality of user-selectable input elements is selected.

5. The emergency response kit of claim 2, wherein the instruction device is a video display device configured to play video instructions regarding use of one of the plurality of equipment kits when a corresponding one of the plurality of user-selectable input elements is selected.

6. The emergency response kit of claim 1, wherein the instruction device is configured to output instructions in a plurality of user-selectable languages.

7. The emergency response kit of claim 1, further comprising:
a tracking device configured to electronically send a location of the emergency response kit to a third party.

8. The emergency response kit of claim 1, further comprising:
a computer-readable medium on which contact information for a plurality of predetermined parties is stored; and
a communication device configured to enable a user to communicate with one of the plurality of predetermined parties in real time, wherein the contact information is selectable by the user to allow the user select one of the plurality of predetermined parties with which to communicate.

9. The emergency response kit of claim 1, wherein the container includes a plurality of written instructions regarding use of the plurality of equipment kits, wherein each of the written instructions include the identifying mark to associate each of the plurality of written instructions with a corresponding one of the plurality of equipment kits.

10. The emergency response kit of claim 1, further comprising:
a mounting element to facilitate mounting the emergency response kit to a desired structure.

11. The emergency response kit of claim 1, further comprising:
a power supply coupled to the emergency response kit to supply power to elements of the emergency response kit requiring power.

12. The emergency response kit of claim 11, wherein power supply is one or more batteries.

13. The emergency response kit of claim 11, wherein the power supply is a solar panel.

14. An emergency response kit, comprising:
a box having an interior volume;
a plurality of containers disposed within the interior volume, wherein each container contains equipment tailored to a predetermined emergency scenario, and wherein each container has a color that is different from colors of other ones of the plurality of containers; and
an audio device configured to output audio instructions regarding use of the equipment inside of the plurality of containers, wherein the audio device includes a plurality of pressable buttons, wherein the plurality of pressable buttons each have a color corresponding to a color of one of the plurality of containers, and wherein upon selection of a pressable button, the audio device plays audio instructions regarding use of the equipment inside one of the plurality of containers associated with the pressable button.

15. The emergency response kit of claim 14, further comprising:
a video display device configured to play video instructions regarding use of one of the equipment inside one of the plurality of containers when a corresponding one of the plurality of pressable buttons is selected.

16. The emergency response kit of claim 14, wherein the audio device is configured to play audio instructions in a plurality of user-selectable languages.

17. The emergency response kit of claim 14, further comprising:
a tracking device configured to electronically send a location of the emergency response kit to a third party.

18. The emergency response kit of claim 14, further comprising:
a computer-readable medium on which contact information for a plurality of predetermined parties is stored; and
a communication device configured to enable a user to communicate with one of the plurality of predetermined parties in real time, wherein the contact information is selectable by the user to allow the user select one of the plurality of predetermined parties with which to communicate.

19. The emergency response kit of claim 14, wherein the box includes a plurality of written instructions regarding use of the equipment inside of the plurality of containers, wherein each of the written instructions include the same color as a corresponding one of the plurality of containers to associate each of the plurality of written instructions with the corresponding one of the plurality of containers.

20. A method of using an emergency response kit, comprising:
opening the emergency response kit;
selecting one of a plurality of equipment kits disposed within the emergency response kit based on an emergency;
pressing a button on an audio device of the emergency response kit corresponding to the selected equipment kit, wherein upon pressing of the button, the audio device plays audio instructions regarding use of equipment inside of the selected equipment kit; and
using equipment provided in the selected equipment kit per the audio instructions to treat a victim of the emergency.
